(19) United States
(12) Patent Application Publication
(10) Pub. No.: US 2020/0046926 A1
(43) Pub. Date: Feb. 13, 2020

(54) Artificial Airway Device

(71) Applicant: Teleflex Life Sciences Unlimited Company, Hamilton (BM)

(72) Inventor: Kien Chung Kwok, Singapore (SG)

(73) Assignee: Teleflex Life Sciences Unlimited Company, Hamilton (BM)

(21) Appl. No.: 16/342,939

(22) PCT Filed: Oct. 20, 2017
(86) PCT No.: PCT/EP2017/076919

§ 371 Date: Oct. 7, 2019

(30) Foreign Application Priority Data
Oct. 21, 2016 (GB) 1617855.0

(51) Int. Cl. A61M 16/04

(52) U.S. Cl. CPC A61M 16/0415; A61M 2016/0027; A61M 16/0497; A61M 16/0447

(57) ABSTRACT

An artificial airway device 1 to facilitate lung ventilation of a patient, comprising an airway tube 2 including a lumen 3, a mask 4 at one end of the airway tube, the mask including a backplate 5 and having a peripheral formation 6 capable of forming a seal around the circumference of the laryngeal inlet, the peripheral formation surrounding a hollow interior space or lumen 7 of the mask and the airway tube 2 opening into the lumen of the mask 4, and a connector 8 disposed at the proximal end of the airway tube, the connector including a main bore 9 for passage of gas to the airway tube lumen 3, and a wall 10 defining a circumference and including a plurality of ports 12 to allow passage of gas to the main bore, at least one port 12 being disposed for circumferential rotational movement relative to the main bore 9.

The present invention relates to an improved artificial airway device, and in particular to a laryngeal mask that is suitable for use in treatment of paediatric patients.

For at least seventy years, endotracheal tubes comprising a long slender tube with an inflatable balloon disposed near the tube's distal end have been used for establishing airways in unconscious patients. In operation, the endotracheal tube's distal end is inserted through the mouth of the patient, into the patient's trachea. Once positioned, the balloon is inflated so as to form a seal with the interior lining of the trachea. After this seal is established, positive pressure may be applied to the tube's proximal end to ventilate the patient's lungs. Also, the seal between the balloon and the inner lining of the trachea protects the lungs from aspiration (e.g., the seal prevents material regurgitated from the stomach from being aspirated into the patient's lungs).

Although they have been successful, endotracheal tubes suffer from several major disadvantages. The principal disadvantage of the endotracheal tube relates to the difficulty of properly inserting the tube. Inserting an endotracheal tube into a patient is a procedure that requires a high degree of skill. Also, even for skilled practitioners, insertion of an endotracheal tube is sometimes difficult or not possible. In many instances, the difficulty of inserting endotracheal tubes has tragically led to the death of a patient because it was not possible to establish an airway in the patient with sufficient rapidity. Also, inserting an endotracheal tube normally requires manipulation of the patient's head and neck and further requires the patient's jaw to be forcibly opened widely. These necessary manipulations make it difficult, or undesirable, to insert an endotracheal tube into a patient who may be suffering from a neck injury.

The use of endotracheal tubes in infants can be particularly challenging. Statistics suggest that in general, levels of anaesthesia-related morbidity and mortality are higher in paediatric patients than in adults, as well as in younger compared to older children and this is often due to airway complications, which are more likely in very young infants. Critical events are highest in infants <2 kg [Tay et. al. Paediatr Anaesth 11: 711, 2001]. In paediatric patients the tongue is relatively larger, more commonly leading to airway obstruction than in adult patients. Paediatric patients often have less pulmonary reserve than adults, and require significantly more oxygen intake, thus they are prone to apnoea during direct laryngoscopy. As the posterior commissure is relatively cephalad, the anterior sublaryngeal airway is predisposed to trauma from an ETT and the narrowest portion of the infant airway is the cricoid cartilage, which can lead to resistance after passing an ETT through the cords.

Children recovering from URI (upper respiratory infection) are at increased risk for respiratory complications. For short procedures via mask, the increased risk is minimal. If reactive airways accompany the infection, the effects of URI may last 2-7 weeks. In particular, those who already have asthma, bronchopulmonary dysplasia, sickle cell, or live in a household of smokers are at high risk, suggesting a “two hit” phenomena [Tait et. al. Anesthesiology 95: 299, 2001]. Bronchial hypereactivity may last as long as 7 weeks after URI [Collier et. al. Am Rev Resp Dis 117: 47, 1978]. Note that in these patients mask anaesthetics have significantly lower complications than an ETT.

If an ETT is required, the risk of anaesthesia in an infant can be increased as much as 10-fold when compared to an infant with no URI and which does not require use of an ETT. Risk of using an LMA are about halfway between those of a facemask and an ETT.

The laryngeal mask airway device is a well known device that is useful for establishing airways in unconscious patients, and which seeks to address some of the known drawbacks associated with endotracheal tubes.

In contrast to the endotracheal tube, it is relatively easy to insert a laryngeal mask airway device into a patient and thereby establish an airway. Also, the laryngeal mask airway device is a “forgiving” device in that even if it is inserted improperly, it still tends to establish an airway. Accordingly, the laryngeal mask airway device is often thought of as a “life saving” device. Also, the laryngeal mask airway device may be inserted with only relatively minor manipulation of the patient's head, neck and jaw. Further, the laryngeal mask airway device provides ventilation of the patient's lungs without requiring contact with the sensitive inner lining of the trachea and the internal diameter of the airway tube is typically significantly larger than that of the endotracheal tube. Also, the laryngeal mask airway device does not interfere with coughing to the same extent as endotracheal tubes. Largely due to these advantages, the laryngeal mask airway device has enjoyed increasing popularity in recent years.

U.S. Pat. No. 4,509,514 describes a laryngeal mask airway device which consists of the basic parts which make up most if not all laryngeal mask airway devices, namely an airway tube opening at one end into the interior of a hollow mask portion shaped to fit readily behind the larynx of a patient. The periphery of the mask is formed by a cuff which in use forms a seal around the opening of the larynx. This enables the airway to be established effectively.

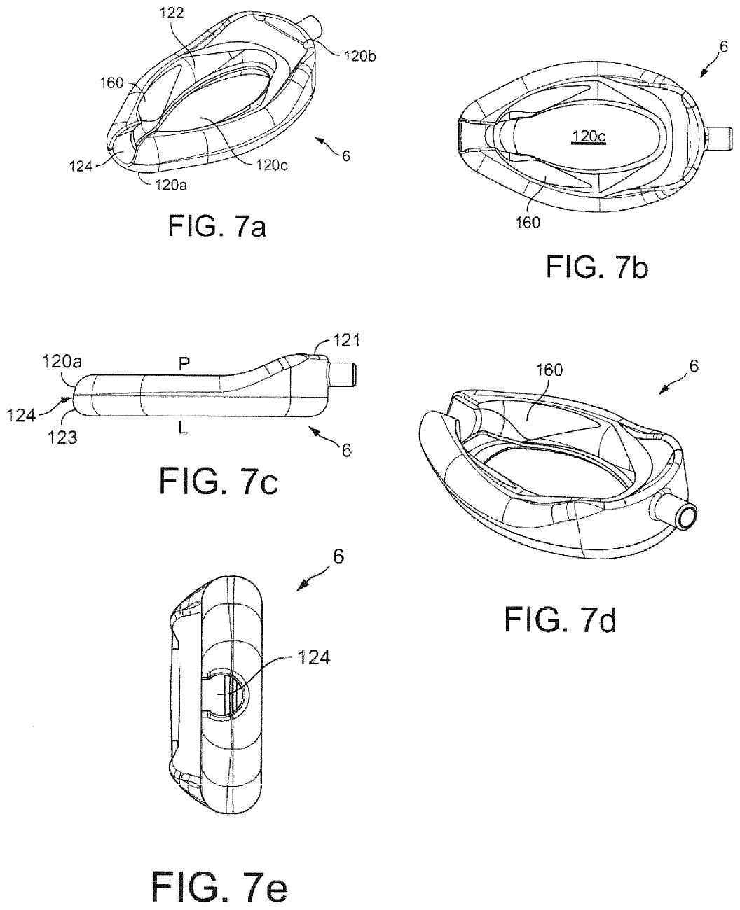

Laryngeal mask airway devices with specific provision for gastric-discharge drainage have been developed, as exemplified by U.S. Pat. No. 4,995,388 (FIGS. 7 to 10); U.S. Pat. Nos. 5,241,956; and 5,355,879. These devices generally incorporate a small-diameter drainage tube having an end located at the distal end of the mask, so as to lie against the upper end of the upper oesophageal sphincter when the mask is in place, the tube being of sufficient length to extend out of the mouth of the patient to enable active or passive removal of gastric discharge from the upper oesophageal sphincter. According to alternative proposals, the drainage tube may extend beyond the distal end of the mask, into the oesophagus itself (U.S. Pat. No. 4,995,388, FIGS. 7 and 11).

Laryngeal mask airway devices are now commonly used to aid in insertion of endotracheal tubes, and such devices are referred to as intubating laryngeal masks, an example being Applicant's own “Fastrach”™ device.

The present invention seeks to ameliorate problems associated with the prior-art described above.

According to a first aspect of the invention there is provided an artificial airway device to facilitate lung ventilation of a patient, comprising an airway tube including a lumen, a mask at one end of the airway tube, the mask including a backplate and having a peripheral formation capable of forming a seal around the circumference of the laryngeal inlet, the peripheral formation surrounding a hollow interior space or lumen of the mask and the airway tube lumen opening into the lumen of the mask, and a connector disposed at the proximal end of the airway tube, the connector including a main bore for passage of gas to the airway tube lumen, and a wall defining a circumference and including a plurality of ports to allow passage of gas to the main bore, at least one port being disposed for circumferential rotational movement relative to the main bore. As will be appreciated, the invention thus provides a device that has numerous advantages. These include, that an air supply can be connected to the device from any desired position relative to the patient's face; the position of the air supply tube relative to the user's face can be moved once it is attached to allow access by the clinician; and the position of the device in the patient is not disturbed by movement of the air supply tube. These advantages are all of particular importance when treating paediatric patients.

It is preferred that the main bore includes a longitudinal axis and that the port that is disposed for circumferential rotational movement includes an inlet that is not coaxial with the longitudinal axis of the main bore. It is further preferred that the main bore includes a proximal end and a distal end, and that said inlet has an axis that is angled towards the proximal end. It is further preferred that the inlet has an axis that is angled toward the proximal end from 30 to 45 degrees to the axis of the main bore.

It is preferred that the main bore includes a longitudinal axis and at least one port that includes an inlet that is coaxial with the longitudinal axis of the main bore. The coaxial inlet may include closure means to close off access to the main bore via the inlet. The closure means may include access means to allow for insertion of instrumentation through the closure into the bore while substantially avoiding escape of gas from the bore. The access means may comprise a pierceable diaphragm.

It is preferred that the connector comprises first and second cylindrical parts, the parts being connected to define the main bore such that each part is rotatable with respect to the other about a common longitudinal axis. It is further preferred that a male section of one cylindrical part is received within a female section of the other cylindrical part, the parts including a mutually inter engageable ridge and groove.

It is preferred that the connector includes a connector plate and an insert, the insert being received within a recess of the airway tube, the connector plate forming an end plate to close off the recess.

It is preferable that the device of the present invention is sized for use in paediatric patients.

It is preferred that at least one port is a gas supply port and that the said gas supply port includes means to reduce the internal volume of the port. The internal volume reduction means may comprise an insert in the bore of the port. The insert may comprise a cylindrical insert disposed within the bore such that fluid flow through the port occurs only through the insert, the external dimensions of the port being unaffected so that connection of devices or fluid flow lines can still be accomplished. This is advantageous because it reduces the dead space in the air supply system which is particularly important for paediatric patients.

The device may further comprise fixation means for fixation of the device to a patient when the device is in use, the fixation means being movable with respect to the airway tube to allow for correct positioning of the device with respect to the anatomy of the patient. It is preferred that the fixation means is disposed on the connector plate.

It is preferred that the fixation means is movably attached relative to the airway tube by first hinge means. It is further preferred that the fixation means includes a plurality of hinges. The provision of a plurality of hinges, and thus a plurality of articulation points means that a precise fit of the device to the patient can be established, which is particularly important in paediatric patients.

It is preferred that the airway tube of the device comprises an outer tube part and an inner core, the inner core defining the airway lumen. The inner core may further define one or more additional lumen adapted to receive a sensor or viewing device. The artificial airway device may further include a conduit disposed to allow in use, for access to the oesophageal sphincter of the patient, and the conduit may be defined by the inner core, or a combination of the inner core and the outer tube part.

Preferably, the sensor is a temperature sensor. Preferably, the temperature sensor comprises a thermistor. Typically, the temperature sensor may be provided on the airway tube. In one embodiment, the temperature sensor may be provided on the inner core. In another embodiment, the temperature sensor may be provided on the outer tube part. In one embodiment the temperature sensor comprises a sensor tip, a lead wire and a connector, wherein the connector may be a moulded connector. Typically, temperature display and logging are achieved by plugging the connector part of the temperature sensor into a patient monitor. In one embodiment, the sensor tip may be encased within the wall of the airway tube along the tube anterior surface. Typically, the sensor tip is encased within the wall of the airway tube along the anterior surface that rests against the pharyngeal portion of the tongue when the device is inserted within a patient. Preferably, the temperature sensor measures the temperature within the orthopharynx of the patient. In one embodiment, the lead wire of the temperature sensor runs along the airway tube, extends out of the airway connector and terminates at the sensor connector. Advantageously, the temperature sensor may be used to measure patient core temperature.

In one embodiment, the device of the present invention may be used with an endotracheal tube.

It is preferred that the peripheral formation comprises an inflatable cuff, or a non-inflatable cuff. It is further preferred that where the peripheral formation comprises an inflatable cuff, the backplate overlies the cuff and is bonded to it, such that on deflation the cuff may be collapsed upon it, thereby encouraging the cuff to pack flat.

According to a second aspect of the invention there is provided a method of treating a patient using a device as defined hereinabove.

The invention will now further be described by way of example, with reference to the accompanying drawings, in which:

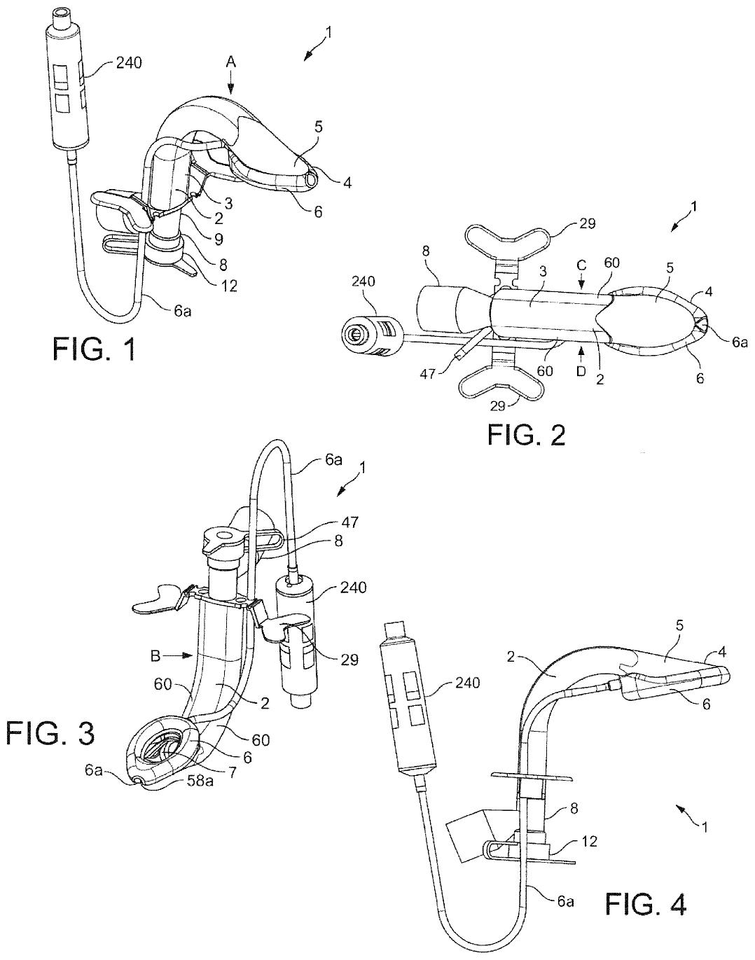

FIG. 1 is a dorsal isometric view of a device according to the invention;

FIG. 2 is a dorsal view of the device of FIG. 1;

FIG. 3 is a ventral isometric view of the device of FIG. 1;

FIG. 4 is a left side view of the device of FIG. 1;

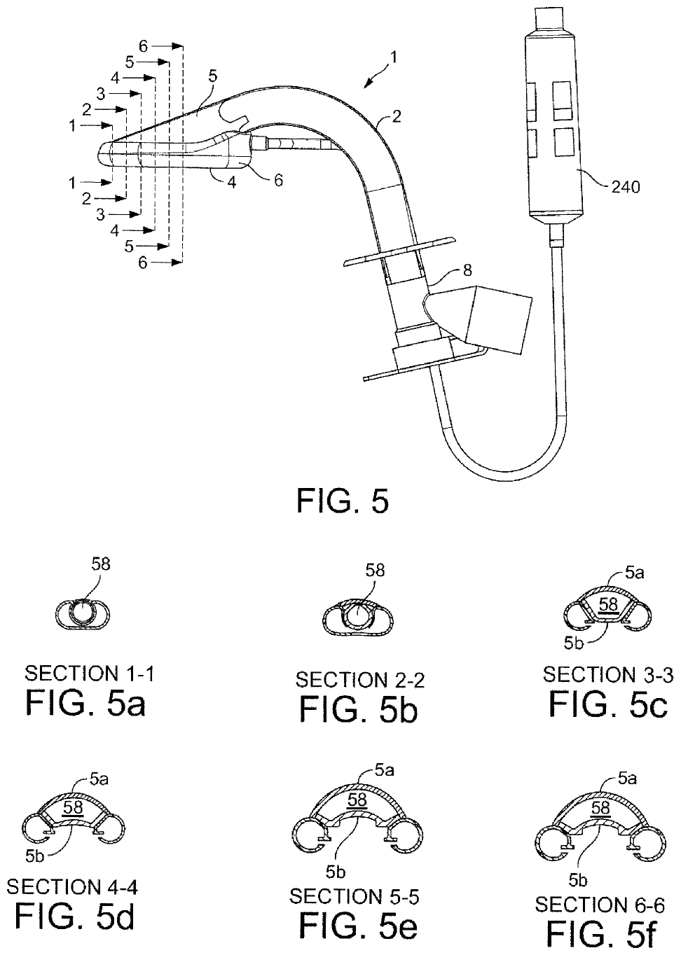

FIG. 5 is a right side view of the device of FIG. 1;

FIGS. 5a to 5f are transverse sectional views along long lines 1-1 to 6-6 in FIG. 5;

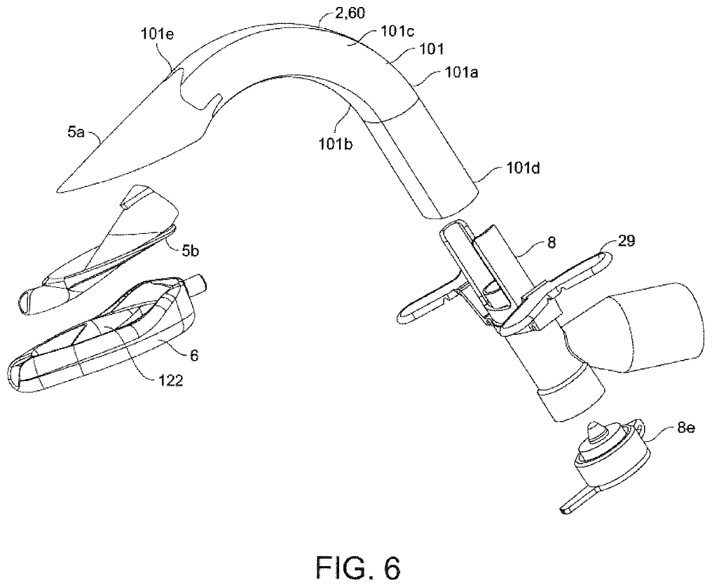

FIG. 6 is a right side exploded view of the device of FIG. 1;

FIG. 7a is a front isometric view of a part of the device of FIG. 1;

FIG. 7b is a dorsal view of the part shown in FIG. 7a;

FIG. 7c is a right side view of the part shown in FIG. 7a;

FIG. 7d is a rear isometric view of the part shown in FIG. 7a;

FIG. 7e is a front view of the part shown in FIG. 7a;

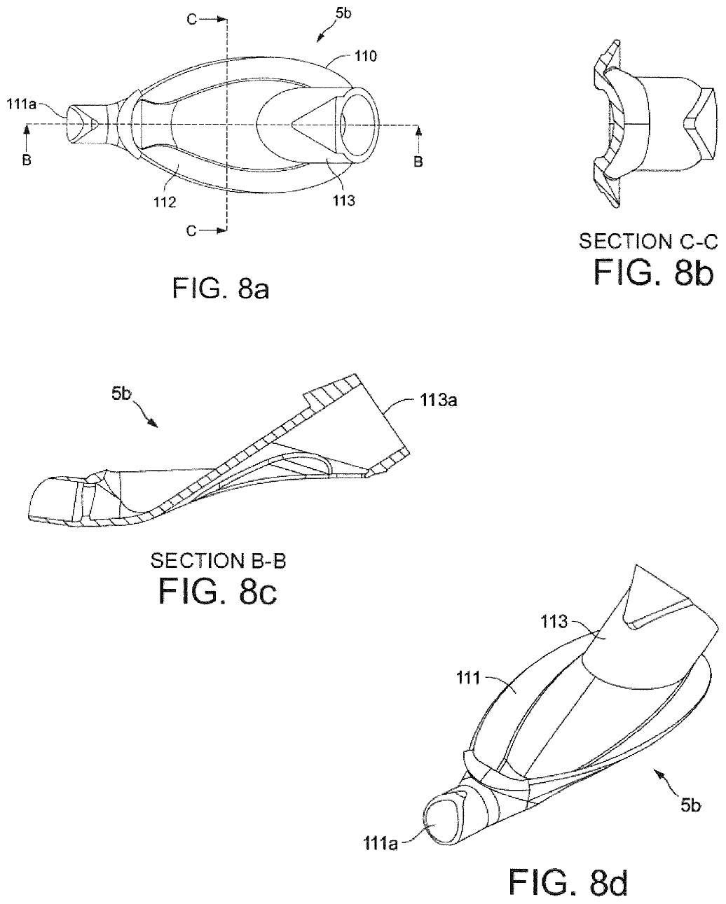

FIG. 8a is a dorsal view of a further part of the device of FIG. 1;

FIG. 8b is a transverse sectional view along line C-C in FIG. 8a;

FIG. 8c is a longitudinal sectional view along line B-B in FIG. 8a;

FIG. 8d is a front dorsal isometric view of the part shown in FIG. 8a;

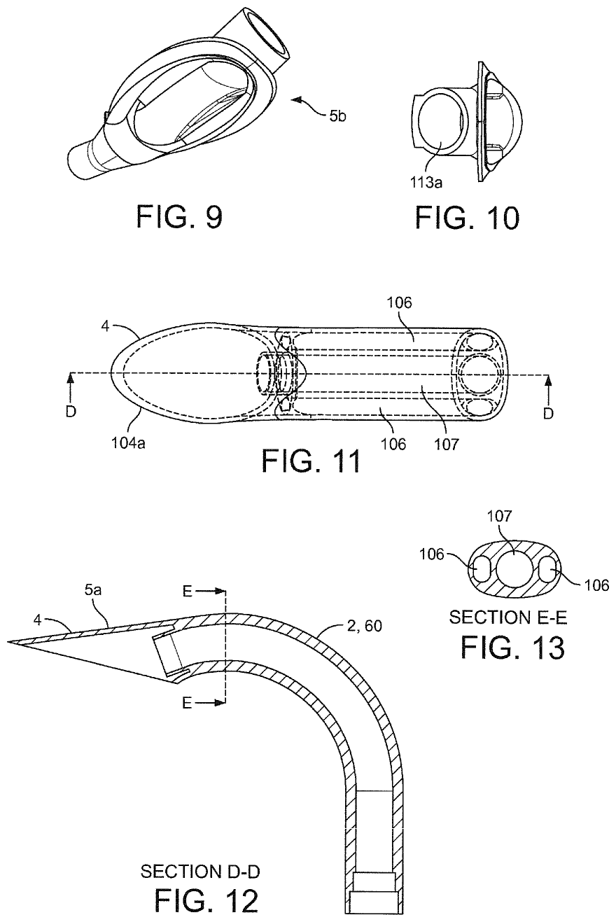

FIG. 9 is a rear ventral isometric view of the part shown in FIG. 8a;

FIG. 10 is a rear view of the part shown in FIG. 8a;

FIG. 11 is dorsal view of a yet further part of the device shown in FIG. 1;

FIG. 12 is a longitudinal sectional view along line D-D in FIG. 11;

FIG. 13 is a transverse sectional view along line E-E in FIG. 12;

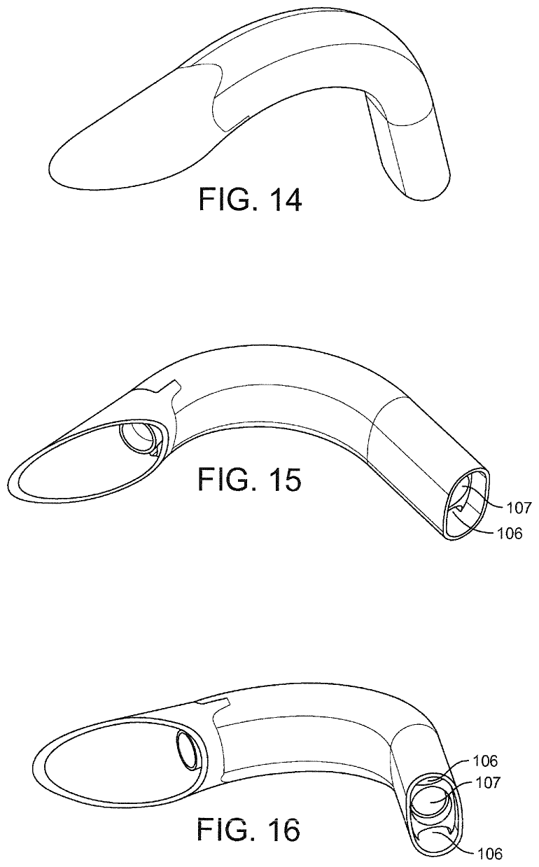

FIG. 14 is a front dorsal isometric view of the part shown in FIG. 11;

FIG. 15 is a right side ventral isometric view of the part shown in FIG. 11;

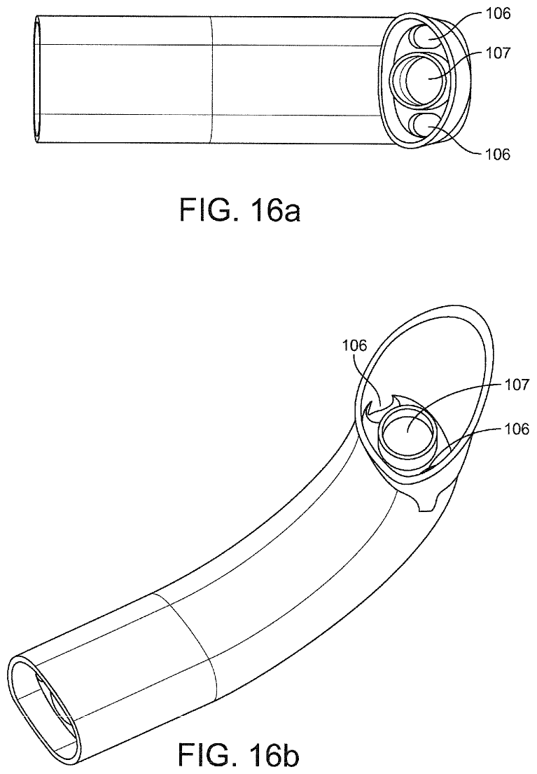

FIG. 16 is a ventral isometric view of the part shown in FIG. 11;

FIG. 16a is a ventral view of the part shown in FIG. 11;

FIG. 16b is a left side ventral isometric view of the part shown in FIG. 11;

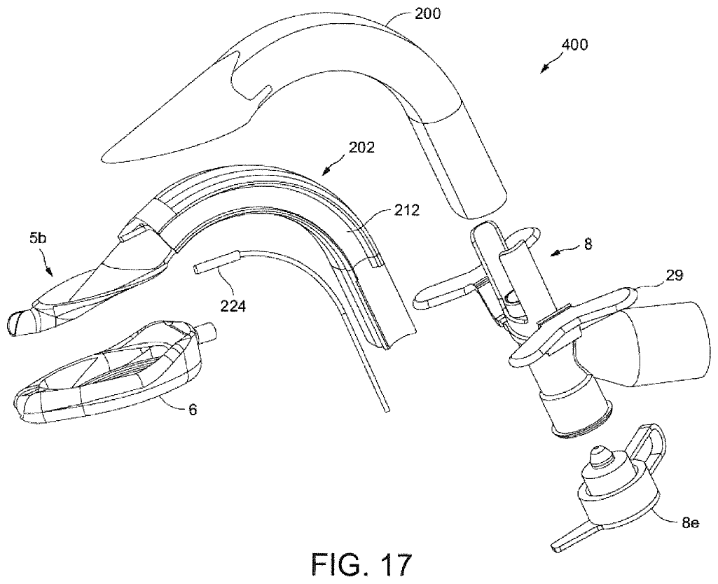

FIG. 17 is a right side exploded view of a second embodiment of device according to the invention;

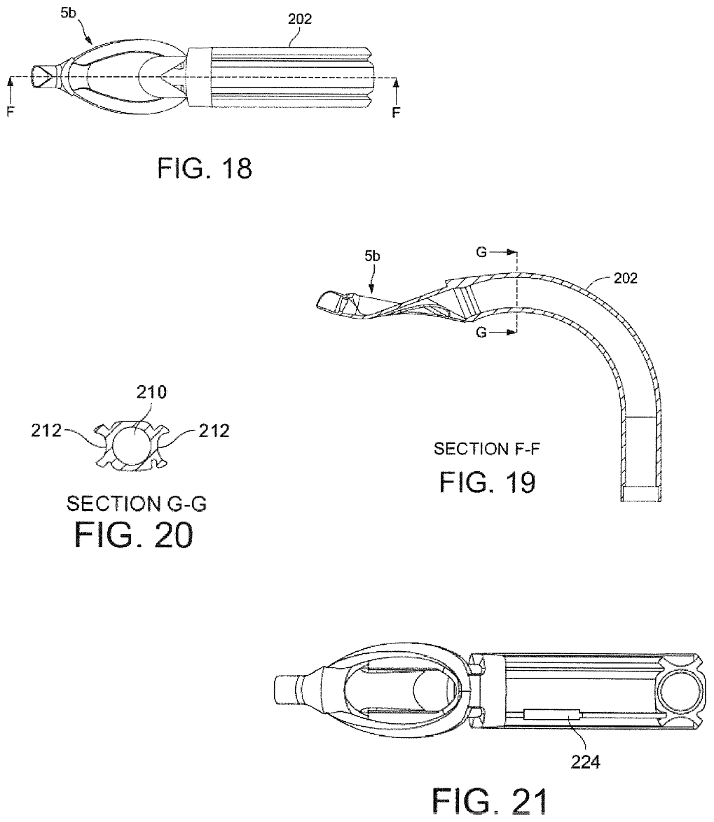

FIG. 18 is a dorsal view of a part of the device shown in FIG. 17;

FIG. 19 is a longitudinal sectional view along line F-F in FIG. 18;

FIG. 20 is a transverse sectional view along line G-G in FIG. 19;

FIG. 21 is a ventral view of the part shown in FIG. 18;

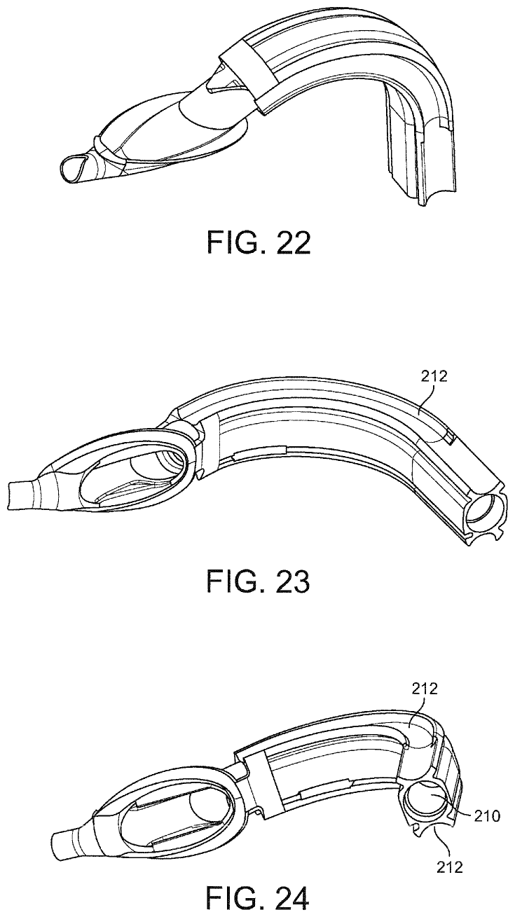

FIG. 22 is a front dorsal isometric view of the part shown in FIG. 18;

FIG. 23 is a right side ventral isometric view of the part shown in FIG. 18;

FIG. 24 is a ventral view of the part shown in FIG. 18;

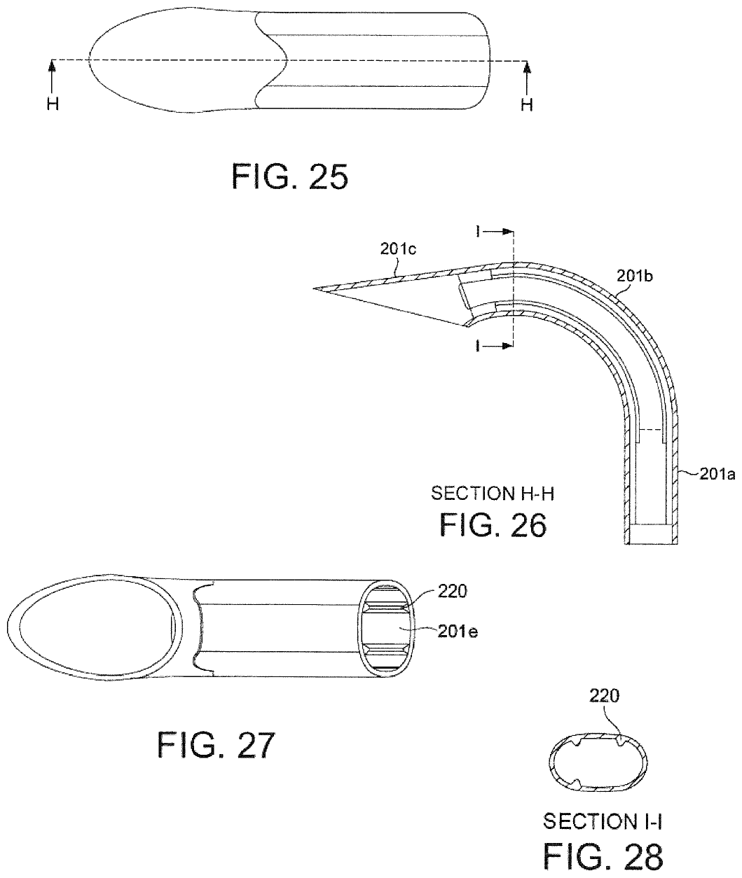

FIG. 25 is a dorsal view of a further part of the device shown in FIG. 17;

FIG. 26 is a longitudinal sectional view along line H-H in FIG. 25;

FIG. 27 is a ventral view of the part shown in FIG. 25;

FIG. 28 is a transverse sectional view along line I-I in FIG. 26;

FIG. 29 is a front dorsal isometric view of the part shown in FIG. 25;

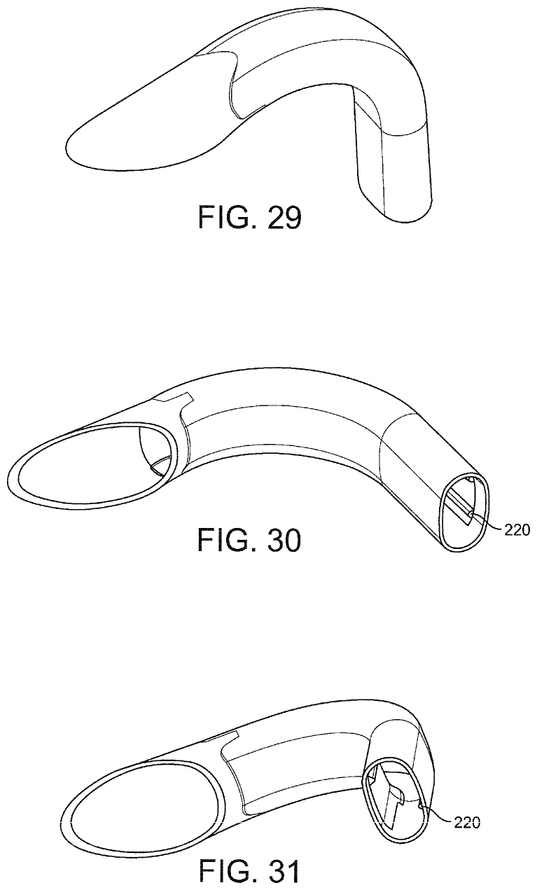

FIG. 30 is a right side ventral isometric view of the part shown in FIG. 25;

FIG. 31 is a right side rear ventral isometric view of the part shown in FIG. 25;

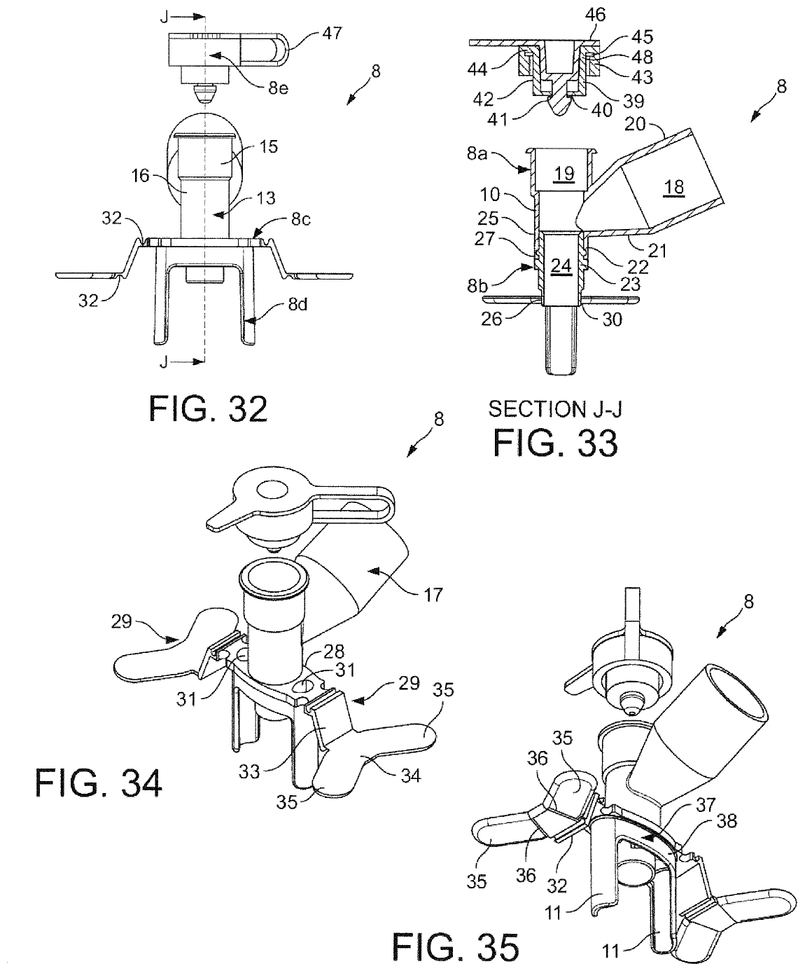

FIG. 32 is a front view of the connector shown in FIGS. 6 and 17;

FIG. 33 is a longitudinal sectional view along line J-J in FIG. 32;

FIG. 34 is a top plan isometric view of the connector shown in FIGS. 6 and 17; and

FIG. 35 is an under plan isometric view of the connector shown in FIGS. 6 and 17.

In the discussion of the following exemplary embodiments, like parts will generally be given the same reference numerals throughout the description.

For convenience of exposition, referring to FIGS. 1 to 4, reference letter A denotes the dorsal surface of the device. Reference letter B denotes the ventral surface of the device. In accordance with standard practice, that part of the device 1 that in use will extend from the patient is referred to herein as the proximal end (in the sense that it is nearest the user) with the other end being referred to as the distal end. In FIG. 2, reference letter C denotes the right side and reference letter D denotes the left side.

With reference to FIGS. 1 to 5, there is illustrated an artificial airway device 1 to facilitate lung ventilation of a patient, comprising an airway tube 2 including an airway tube lumen 3, a mask 4 at one end of the airway tube, the mask including a backplate 5 and having a peripheral formation 6 capable of forming a seal around the circumference of the laryngeal inlet, the peripheral formation surrounding a hollow interior space or lumen 7 of the mask and the airway tube 2 opening into the lumen of the mask 4, and a connector 8 disposed at the proximal end of the airway tube, the connector including a main bore 9 for passage of gas to the airway tube lumen 3, the main bore including a wall 10 defining a circumference and including a plurality of ports 12 to allow passage of gas to the main bore, at least one port 12 being disposed for circumferential rotational movement about the main bore 9.

Connector 8 is illustrated in detail in FIGS. 32 to 35. Referring to FIGS. 32 and 33, connector 8 comprises five parts, namely access port part 8a, main bore part 8b, fixation part 8c, insert part 8d and plug 8e. With the exception of the plug 8e, each part may be injection moulded from polypropylene or polyethylene. Plug 8e is preferably formed from silicone by liquid injection moulding, transfer moulding or compression moulding.

Access port part 8a comprises a main tube 13 including a generally cylindrical wall 10 having a bore 19 and respectively an outer larger diameter part 15, an inner smaller diameter part 16, and a branch tube 17. Branch tube 17 defines branch bore 18 and is attached to inner smaller diameter part 16 such that branch bore 18 is in fluid communication with bore 19. Branch tube 17 includes an outer constant diameter section 20 that is dimensioned to connect to a standard gas supply. Constant diameter section 20 is connected to a frustoconical section 21 that in turn connects to wall 10. Inner smaller diameter part 16 includes inner circumferential groove 22 adjacent the distal end.

Main bore part 8b comprises a tubular wall 23 defining a bore 24 and proximal and distal ends 25, 26. Proximal end 25 is dimensioned to be received within bore 19 of access port part 8a and includes outer circumferential ridge 27 that is dimensioned to fit into inner circumferential groove 22 of access port part 8a.

Fixation part 8c comprises generally rectangular plate 28, and fixation tabs 29. Plate 28 includes a central through-bore 30 and two side through-bores 31 which extend between the major surfaces of the plate. Fixation tabs 29 extend from the minor end surfaces of the plate 28, and are hingedly attached thereto by webs 32. Each fixation tab 29 comprises a connector plate 33, a lower plate 34 and tabs 35. As viewed in FIGS. 32 to 35 and when in use in a patient, connector plate 33 depends downwardly from its proximal hinged attachment point at a minor end surface of plate 28 at a resting angle of greater than 90 degrees thereto. At its distal end each connector plate is further hingedly attached to a lower plate 34, the surface of which is disposed at rest substantially parallel to, but at a lower level than, the surface of plate 28. Each lower plate 34 comprises two tabs 35 which are co-planar with plate 34 at rest and hingedly attached thereto via hinge points 36 (FIG. 35).

Referring to FIG. 35, insert part 8d comprises an ellipsoidal mounting ring 37 having a circumferential wall 38 and depending legs 11. Each depending leg 11 comprises an arcuate wall.

Referring to FIG. 33, plug 8e comprises a circular cup insert 39 that is dimensioned to fit via an interference fit into bore 19 of access port 8a. Insert 39 includes a bottom surface 40 with a centrally disposed through-bore 41 and a circumferential wall 42. Wall 42 includes a circumferential skirt 43 depending from its upper, as viewed edge 44, thereby defining a downwardly open channel 45 between skirt and wall. Plug 8e further comprises cap 46 which is attached by retaining strap 47 to skirt 43 and is dimensioned to fit within cup insert 39. Cap 46 includes depending knob 48 which fits within through-bore 41 when the cap is in place in the plug.

Referring in particular to FIG. 33, the parts are assembled by firstly connecting parts 8a and 8b by a push fit. Part 8b proximal end 25 is received within bore 19 of access port part 8a such that outer circumferential ridge 27 fits into inner circumferential groove 22. The ridge and groove ensure that parts 8a and 8b are held together, but they can rotate with respect to on another. This has the effect that the position of the branch tube 17 can be rotated around 360 degrees relative to the main bore. The plug component 8e of the connector comprises a circular cup insert 39 that is dimensioned to fit via an interference fit into bore 19 of access port 8a. The plug 8e is attached by a retaining strap 47 to skirt 43 and is dimensioned such that it fits within cup insert 39. Cap 46 including a depending knob 48 fits within through bore 41 when the cap is in place in the plug.

The connector 8 is inserted into the proximal end of the airway tube by inserting the insert part 8d into a recess provided at the end of the airway tube 2. The insert part 8d comprises depending legs 11, each depending leg 11 comprising an arcuate wall and being dimensioned such that when the insert part 8d fits within the recess of the airway tube, each leg 11 passes into a respective gastric drainage lumen 106 of the airway tube. At the same time, main bore distal end 26 is received into airway tube airway lumen 3. The insert part of the connector passes through the central through-bore 30 of the fixation part 8c. The fixation part 8c is positioned at the proximal end of the airway tube, wherein the major surface of the plate 28 extends along a length which is substantially perpendicular to the longitudinal axis of the laryngeal mask airway device.

At its distal end, airway tube 2 is attached to mask 4. Airway tube 2 and mask 4 may be formed integrally or separately. It will be noted that airway tube 2 terminates towards the proximal end of mask 4. Thus mask 4 does not suffer in terms of being made too rigid by the material of the airway tube. One notable feature of the present invention is the construction of the backplate 5. As the skilled worker will appreciate, the term “backplate”, when used in the present technical field has come to denote that part of the mask that is surrounded by the cuff in the assembled device and which provides separation between the laryngeal and pharyngeal regions when the device is in situ in the patient. Supply of gas takes place through an aperture in the backplate via a fluid tight connection between the part of the backplate defining the aperture and the airway tube. In one known arrangement the backplate and airway tube are formed integrally which is a particularly convenient arrangement. In the prior art, backplates are generally bowl or dome shaped structures rather than flat structures and the term is therefore not entirely descriptive of the shape.

The device further comprises a component 240 for monitoring the pressure of the cuff to check that the cuff has been inflated correctly.

In the embodiment as shown in FIGS. 1 to 5, the device includes a dual gastric drain 60 in the form of a softly pliant sleeve that terminates at its distal end in atrium 58. Thus, the device of FIGS. 1 to 5 comprises two gastric drain tubes 60.

In the presently described embodiment backplate 5 comprises inner and outer skins 5a, 5b that together define a space therebetween, as shown schematically in FIGS. 5a to 5f. The space so defined is atrium 58 from which proximally, drain tubes 60 lead off and distally, inlet 58a enters. The atrium can be regarded as a manifold that connects the single gastric inlet 58a with the gastric drain tubes 60. The gastric drain tubes 60 and backplate may be integrally formed.

Airway tube 2 is formed from a material such that it is not collapsible and has a preformed fixed curve as illustrated in FIG. 1. As an example, the airway tube 2 may be of 80 Shore A durometer according to ASTM 2240. The airway tube may be formed from any known suitable material such as PVC or silicone.

As mentioned above, mask 4 includes peripheral formation 6 which in this embodiment takes the form of an inflatable cuff of generally known form. Cuff 6 includes an inflation line 6a at its proximal end and has a gastric inlet aperture 6b at its distal end (FIG. 3). Referring to the exploded view in FIG. 5, it can be seen that the dorsal surface of cuff 6 is bonded to backplate 5 so that the material of the dorsal surface of the cuff 6 forms a bridge between the inner and outer skins 5a, 5b thus closing off the ventral side of atrium 58 except where gastric inlet aperture 6b enters the cuff. Thus it can be seen that gastric inlet 6b is in fluid communication with atrium 58. In an alternative method of construction the cuff 6 may be formed with a web across its aperture that itself forms the ventral surface of atrium 58.

In use, the device 1 is inserted into a patient to establish an airway as with prior art devices. Insertion is effected to the point where gastric inlet aperture 6b meets the patient's oesophageal sphincter, thus establishing fluid communication therebetween. If vomiting or regurgitation occurs, as with previous gastric access laryngeal masks, the material from the oesophagus passes into gastric inlet aperture 6b. However, unlike with previous devices the material passes into the atrium 58 formed between the dual backplate skins 5a 5b, the volume of which is larger than the volume of the inlet aperture 6b. It will be appreciated that constructing a laryngeal mask with a backplate 5 in which is formed an atrium or conduit 58 for gastric material is a highly efficient and economical way to use existing mask structures. Forming gastric drain tubes from an expandable material so that the space they occupy in the anatomy is minimised until they are called upon to perform their function is advantageous because it makes insertion of the device easier and causes less trauma to the delicate structures of the anatomy when the device is in place, particularly if the device is left in place for an extended period. And still further advantages are obtained if these features are combined such that the atrium 58 is formed from the soft material of the gastric drain tubes makes because the mask, whilst being sufficiently soft to avoid trauma on insertion can yet provide a large volume atrium 58 that can expand under pressure of vomiting. Such expansion results in a dorsal deformation of the outer skin 5b resembling a dome that acts like a spring against the back wall of the throat when the mask is in situ, forcing the cuff 6 against the larynx and thereby helping to maintain the device in its sealed state. The use of the device comprising connector 8 has the advantages that an air supply can be connected to the device from any desired position relative to the patient's face, the position of the air supply tube can moved once it is attached to allow access by the clinician, and the position of the device in the patient is not disturbed by movement of the air supply. The use of a device comprising fixation straps allows the device to be positioned very precisely by virtue of the hinges which provide multiple points of articulation and allow the position and degree of insertion to be tailored precisely to the patient's anatomy.

FIG. 6 shows an exploded view of the device of FIGS. 1 to 5 to demonstrate how the parts of the device are fitted together. From the exploded view of FIG. 6 it can be seen that the device 1 comprises three main parts, a gastric drain and airway tube and backplate combination part 2, 60, 5a; an inner backplate wall 5b, and a peripheral formation 6, as well as the connector 8. It can be seen that the outer backplate part 5a, and inner backplate wall 5b are combined to form the backplate 5, thus defining a conduit in the form of chamber or atrium 58 within the backplate 5.

The peripheral part 6, in this embodiment an inflatable cuff, is attached to the backplate 5 by bonding to the attachment surface 122 such that the backplate 5 seats within it.

The gastric drain and airway tube and backplate combination part 2, 60, 5a consists of a precurved tube 101. The tube 101 is not circular in cross-section but has a flattened section, as taught in previous patents, for ease of insertion and fit through the interdental gap. The tube 101 has flattened dorsal and ventral surfaces 101a, 101b and curved side walls 101c extending from a proximal end 101d to a distal end 101e. At its distal end the combination part 2, 60, 5a is cut at an angle relative to its longitudinal axis to provide an outer backplate part 5a which may be integrally formed therewith, for example by molding. As an alternative the outer backplate part 5a can be separately formed, for example, from a transparent or translucent material. The outer backplate part 5a may include a circumferential lip. Finally, with reference to FIG. 11, it will be noted that gastric drain, airway tube and backplate combination part includes a substantially coaxially disposed inner tube extending from the distal end to the proximal end, the inner tube effectively establishing a separation of the inner space into two gastric conduits 106 and an airway conduit 107. This arrangement is further illustrated in FIGS. 12 and 13 and 14 to 16b, wherein FIG. 12 shows the view through Section D-D of FIG. 11 and FIG. 13 shows the view through Section E-E of FIG. 12.

Referring now to FIGS. 8a to 8d and FIGS. 9 and 10, there is illustrated inner backplate wall 5b. Inner backplate wall 5b comprises a generally elliptical body in the form of a shallow dish including side wall 111 and floor 112. At the distal, or narrower end of the elliptical dish, side wall 111 has a cylindrical aperture 111a formed therein that extends distally generally in line with the midline of the floor 112. It will be noted that cylindrical aperture 111a may be angled upwardly, relative to the plane of the floor 112 such that the angle of the axis of the bore of the cylindrical aperture is about 20 degrees relative thereto. Along its midline the floor 112 of the dish is raised to form a convex surface that extends longitudinally towards the wider, proximal end where it terminates in a cylindrical formation that may be referred to as a tube joint 113. Tube joint 113 includes bore 113a that provides a connecting passage between the upper and lower surfaces (as viewed) of floor 112. Tube joint 113 merges with and bisects side wall 111 and is angled upwardly at about 45 degrees relative to floor 112, terminating proximally some distance beyond the side wall 111 as shown in FIG. 9.

Referring now to FIGS. 7a to 7e, there is illustrated peripheral formation 6 which in this embodiment takes the form of an inflatable cuff. It will be noted that unlike many other laryngeal mask airway devices the cuff 6 is formed integrally as a separate part from the rest of the device, making it easier both to manufacture and attach to the device 1. The cuff 6 comprises a generally elliptical body with a narrower distal end 120a, a wider proximal end 120b and a central elliptical through-aperture 120c. As such it will be appreciated that the cuff resembles a ring. As can be seen from the sectional view in FIG. 7c, the elliptical body comprises a wall 123 that is generally circular in section at the distal end but deeper and irregularly shaped at the proximal end by virtue of an integrally formed extension 121 formed on the dorsal surface at the proximal end 120b. This dorsal surface extension 121 defines the proximal portion of an attachment surface 122 (FIGS. 6 and 7a). The attachment surface 122 extends from the proximal end to the distal end around the entire dorsal inner circumference of the ring. At its distal end 120a the cuff has a cylindrical through bore 121 the axis of which extends in line with the midline of the ellipse and is angled upwardly as viewed in FIG. 7c relative to the plane of the body, in other words from the ventral towards the dorsal side or when the device 1 is in use from the laryngeal to the pharyngeal side of the anatomy (L and P in FIG. 7c). The result is a circular section aperture through the cuff wall 123. The proximal end 120b of the cuff includes a port 124 that lets into the interior of the bore and the cuff. As illustrated, for example, in FIGS. 7a, 7b and 7d, the cuff comprises side projections 160 which help to prevent the occlusion of the airway by supporting the anatomy of the patient.

Thus, in this embodiment, the airway tube, gastric drain and backplate combination part comprises the airway tube and the gastric drain tubes. It has been found that contrary to expectation it is most important in a device having a gastric tube that flow of gastric material should not be impeded, so that the seal formed around the upper oesophageal sphincter is not broken. This arrangement best utilises the available space within the anatomy to achieve this end. Similarly, the provision of an atrium 58 to receive gastric flow as opposed to the simple uniform section conduits of prior devices provides a mask that is in effect a hollow leak-free plug against the upper oesophageal sphincter, with a low-flow high-volume escape route above it. The device 1 of this embodiment of the invention enables a user to get such a plug into place and hold it there whilst providing a sufficiently generous escape path for emerging fluids. Further still, it has been found that the provision of a gastric inlet port that is angled dorsally as described further aids in ensuring that the seal around the upper oesophageal sphincter remains intact even under heavy load, particularly when an atrium is provided directly upstream therefrom.

Referring now to FIGS. 17 to 31, there is illustrated a further alternative embodiment of device 400 according to the invention. This embodiment differs from the previously described embodiment in a number of important respects as will be described. However it will be appreciated that the concepts which it embodies may be applied to the previously described embodiments and vice versa.

With reference to FIG. 17, it can be seen that the device 400 resembles other laryngeal mask airway devices. In the embodiment of FIG. 17 it can be seen that the device includes an airway tube 2 that comprises what is in effect, an airway tube and backplate combination part 200. The airway tube and backplate combination part 200 comprises two pieces: an outer tube 201 and an inner core 202.

Outer tube is illustrated in detail in FIGS. 25 to 27. From these it can be seen that the outer tube takes the form of a tube having a straight portion 201a, a fixed curve portion 201b and a backplate portion 201c moving from the proximal to distal end. In transverse section the tube is compressed rather than circular (FIG. 28) as is known in the art, with a through bore 201d running throughout from its proximal to distal ends. As illustrated, for example, in FIG. 27, the inner surface 201e of the sheath 201 comprises three raised tracks 220 which extend from near the proximal end to the distal end of the straight portion 201a, one on the ventral inner surface and two on the opposing dorsal surface.

As mentioned, at its distal end outer tube 201 includes backplate portion 201c. One notable feature of the present invention is the construction of the backplate. As the skilled worker will appreciate, the term “backplate” when used in the present technical field has come to denote that part of the mask that is surrounded by the cuff in the assembled device and which provides separation between the laryngeal and pharyngeal regions when the device is in situ in a patient. Supply of gas takes place through an aperture in the backplate via a fluid tight connection between the part of the backplate defining the aperture and the airway tube. In one known arrangement, the backplate and airway tube are formed integrally which is a particularly convenient arrangement. In the prior art, backplates are generally bowl or dome shaped structures rather than flat structures and the term is therefore not entirely descriptive of the shape. In the presently described device, the outer tube 201 provides a part of the backplate, in particular, backplate portion 201c that acts as an outer cover or skin. Thus, the backplate 5 comprises inner and outer skins 5a, 5b that together define a space there between. The space so defined is atrium 58 from which proximally, drain tubes 60 lead off an inlet 58a enters. The atrium can be regarded as a manifold that connects the single gastric inlet with the gastric drain tubes.

Referring now to FIGS. 17 to 24, there is illustrated inner core 202. The inner core 202 is dimensioned to fit inside the outer tube part 201 and typically extends substantially along the entire length of the outer tube part 201. Preferably, the inner core element comprises an inner backplate portion. The inner core 202 comprises a tube and defines partly or completely the airway lumen (see FIG. 20). The inner core 202 further defines partly or completely one or more additional lumen or groove 212. The one or more additional lumen may be adapted to receive a sensor or a viewing device, for example, the additional lumen may include a recess for location of a sensor. The one or more additional lumen may further include one or more lumen to allow in use, for access to the oesophageal sphincter of the patient and/or removal of gastric fluid. The one or more additional lumen may be defined entirely by the inner core 202, or by the combination of the inner core 202 and the outer tube part 201. Thus, the inner core element allows a plurality of conduits to be defined within the airway tube and backplate combination part, allowing for passage of gastric matter, introduction of sensors or viewing devices, etc.

In the embodiment shown in FIGS. 17 and 20, the inner core element comprises two lumens, the lumens extending along the left and right sides of the inner core 202. The lumens may be provided in the form of a groove within the outer surface of the inner core 202. In this embodiment, when the inner core 202 is inserted within the outer tube 201, the combination of the lumens 212 of the inner core element 202 and the inner wall of the outer tube 201 form two gastric conduits for passage of gastric matter.

At least one further groove or recess may be provided on an outer surface of the inner core 202. The at least one further groove corresponds to the at least one track provided on an inner surface of the outer tube part, such that at least one further groove and at least one track engage with one another. In another embodiment, the at least one track is provided on an outer surface of the inner core and the at least one further groove or recess is provided on an inner surface of the outer tube part. The provision of at least one track 220 and at least one further groove provides guide means to facilitate insertion of the inner core 202 within the outer tube part 201 and further provides a means for securing the inner core 202 in place within the outer tube 201 during use of the device.

As shown, for example, in FIG. 21, the inner core 202 may define an additional lumen adapted to receive a sensor or viewing device (224). In one embodiment, the sensor may be a temperature sensor. Preferably, the temperature sensor comprises a thermistor. Typically, the temperature sensor may be positioned on the airway tube. In one embodiment, the temperature sensor may be positioned on the inner core element 202. In another embodiment, the temperature sensor may be positioned on the outer tube part 200. In one embodiment the temperature sensor comprises a sensor tip, a lead wire and a connector, wherein the connector may be a moulded connector. Temperature display and logging are typically achieved by plugging the connector part of the temperature sensor into a patient monitor. In one embodiment, the sensor tip is encased within the wall of the airway tube along the tube anterior surface. Typically, the sensor tip is encased within the wall of the airway tube along the anterior surface that rests against the pharyngeal portion of the tongue when the device is inserted within a patient. Preferably, the temperature sensor measures the temperature within the orthopharynx of the patient. In one embodiment, the lead wire of the temperature sensor runs along the airway tube, extends out of the airway connector and terminates at the sensor connector. Advantageously, the temperature sensor may be used to measure patient core temperature. In one embodiment, the device of the present invention may be used with an endotracheal tube.

The airway tube may be formed by fitting together the inner core 202 and the outer tube 201, wherein the inner core is inserted into the outer tube 202. When the inner core 202 is inserted into the outer tube 201, the inner core provides strength and rigidity to the airway tube and backplate combination part.

From the exploded view of FIG. 17 it can be seen that the device 400 comprises an airway tube and backplate combination part 200, an inner core 202, an outer tube part 201, an inner backplate wall part 5b, a peripheral formation 6, and a connector 8. In this embodiment, the inner core defines an airway lumen 210. The inner core 202 and inner backplate wall 5b may be integrally formed. Alternatively, in another embodiment, the inner core 202 and inner backplate wall 5b may be formed separately and subsequently attached. At least one gastric conduit 260 is defined by the inner core 202, or a combination of the inner core 202 and the outer tube part 200.

The peripheral formation 6 of this embodiment comprises the features as described in previous embodiments.

In use, the device 400 comprising the airway tube and backplate combination part 200 and the inner core 202 is inserted into a patient to establish an airway as with prior art devices. Insertion is effected to the point where gastric inlet aperture 6b meets the patient's oesophageal sphincter, thus establishing fluid communication therebetween. If vomiting or regurgitation occurs, as with previous gastric access laryngeal masks, the material from the oesophagus passes into gastric inlet aperture 6b. However, unlike with previous devices the material passes into the atrium 58 formed between the dual backplate skins 5a 5b, the volume of which is larger than the volume of the inlet aperture 6b. It will be appreciated that constructing a laryngeal mask with a backplate 5 in which is formed an atrium or conduit 58 for gastric material is a highly efficient and economical way to use existing mask structures, as discussed in relation to the embodiments shown in FIGS. 1 to 16. Furthermore, the use of a device comprising an inner core comprising an airway lumen and two gastric lumens provided by the combination of the inner core inserted within the outer tube part allows for efficient air supply to the patient and drainage of gastric matter. The provision of the inner core provides for flexibility of use such that further conduits may be provided for insertion of a sensor or viewing device, as required. The use of the device comprising connector 8 has the advantages that an air supply can be connected to the device from any desired position relative to the patient's face, the position of the air supply tube can moved once it is attached to allow access by the clinician, and the position of the device in the patient is not disturbed by movement of the air supply. The use of a device comprising fixation straps allows the device to be positioned very precisely by virtue of the hinges which provide multiple points of articulation and allow the position and degree of insertion to be tailored precisely to the patient's anatomy.

Thus, it can be seen that the above described embodiments address the problems of prior art devices in novel and inventive ways.

Features of the above-described embodiments may be re-combined into further embodiments falling within the scope of the present invention. Further, the present invention is not limited to the exemplary materials and methods of construction outlined above in connection with the exemplary embodiments, and any suitable materials or methods of construction may be employed. For example, although the cuff may be formed using a sheet of soft flexible silicone rubber, other materials such as latex or PVC may be used. PVC as a material is particularly suited to embodiments intended for single use, whereas the use of silicone rubber is preferred although not essential for embodiments intended to be re-used in a number of medical procedures.

Further, and as would be appreciated by the skilled person, various features of the present invention are applicable to a wide range of different laryngeal mask airway devices, and the invention is not limited to the exemplary embodiments of types of mask described above. For example, aspects of the invention may be applied to laryngeal mask airway devices featuring epiglottic elevator bars over the mask aperture, which bars are operable to lift the epiglottis of a patient away from the aperture upon insertion of an endotracheal tube or other longitudinally-extended element inserted through the airway tube so as to emerge into the hollow or lumen of the mask through the mask aperture. Aspects of the present invention may for example be applied to single or re-useable devices, devices featuring aperture bars or not, “intubating” devices which permit an endotracheal tube or similar to be introduced into the larynx via an airway tube of a mask, devices incorporating fiberoptic viewing devices and so forth, without restriction or limitation on the scope of the present invention.

1. An artificial airway device to facilitate lung ventilation of a patient, comprising an airway tube including a lumen, a mask at one end of the airway tube, the mask including a backplate and having a peripheral formation capable of forming a seal around the circumference of the laryngeal inlet, the peripheral formation surrounding a hollow interior space or lumen of the mask and the airway tube opening into the lumen of the mask, and a connector disposed at the proximal end of the airway tube, the connector including a main bore for passage of gas to the airway tube lumen, and a wall defining a circumference and including a plurality of ports to allow passage of gas to the main bore, at least one port being disposed for circumferential rotational movement relative to the main bore.

2. A device according to claim 1, wherein the main bore includes a longitudinal axis and wherein the port that is disposed for circumferential rotational movement includes an inlet that is not coaxial with the longitudinal axis of the main bore.

3. A device according to claim 1 or 2, wherein the main bore includes a proximal end and a distal end, and wherein the inlet has an axis that is angled towards the proximal end.

4. A device according to claim 2 or 3, wherein the inlet has an axis that is angled toward the proximal end from 30 to 45 degrees to the axis of the main bore.

5. A device according to any preceding claim, wherein the main bore includes a longitudinal axis and at least one port that includes an inlet that is coaxial with the longitudinal axis of the main bore.

6. A device according to claim 5, wherein the coaxial inlet includes closure means to close off access to the main bore via the inlet.

7. A device according to claim 6, wherein the closure means includes access means to allow for insertion of instrumentation through the closure into the bore while substantially avoiding escape of gas from the bore.

8. A device according to claim 7, wherein the access means comprises a pierceable diaphragm.

9. A device according to any preceding claim, wherein the connector comprises first and second cylindrical parts, the parts being connected to define the main bore such that each part is rotatable with respect to the other about a common longitudinal axis.

10. A device according to claim 9, wherein a male section of one cylindrical part is received within a female section of the other cylindrical part, the parts including a mutually inter engageable ridge and groove.

11. A device according to any preceding claim, wherein the connector includes a connector plate and an insert, the insert being received within a recess of the airway tube, the connector plate forming an end plate to close off the recess.

12. A device according to any preceding claim, wherein the device is sized for use in paediatric patients.

13. A device according to any preceding claim, wherein at least one port is a gas supply port.

14. A device according to claim 13, wherein the gas supply port comprises means to reduce the internal volume of the port.

15. A device according to claim 14, wherein the internal volume reduction means comprises an insert in the bore of the port.

16. A device according to claim 15, wherein the insert comprises a cylindrical insert disposed within the bore such that fluid flow through the port occurs only through the insert, the external dimensions of the port being unaffected so that connection of devices or fluid lines can still be accomplished.

17. A device according to any preceding claim, further comprising fixation means for fixation of the device to a patient when the device is in use, the fixation means being movable with respect to the airway tube to allow for correct positioning of the device with respect to the anatomy of the patient.

18. A device according to claim 17, wherein the fixation means is disposed on the connector plate.

19. A device according to claim 17 or 18, wherein the fixation means is movably attached relative to the airway tube by first hinge means.

20. A device according to claim 17, 18 or 19, wherein the fixation means includes a plurality of hinges.

21. A device according to any preceding claim, wherein the airway tube comprises an outer tube part and an inner core, the inner core defining the airway lumen.

22. An artificial airway device according to claim 21, wherein the inner core further defines one or more additional lumen adapted to receive a sensor or viewing device.

23. A device according to claim 21 or 22, further comprising a conduit disposed to allow in use, for access to the oesophageal sphincter of the patient.

24. A device according to claim 23, wherein the conduit is defined by the inner core, or a combination of the inner core and the outer tube part.

25. A device according to claim 22, 23 or 24, wherein the sensor is a temperature sensor.

26. A device according to claim 25, wherein the temperature sensor is a thermistor.

27. A device according to claim 25 or 26, wherein the temperature sensor is provided on the airway tube.

28. A device according to claim 27, wherein the temperature sensor is provided on the inner core.

29. A device according to claim 27, wherein the temperature sensor is provided on the outer tube part.

30. A device according to any one of claims 25 to 29, wherein the temperature sensor comprises a sensor tip, a lead wire and a connector.

31. A device according to any preceding claim, wherein the peripheral formation comprises an inflatable cuff, or a non-inflatable cuff.

32. A device according to claim 31, wherein when the peripheral formation comprises an inflatable cuff, the backplate overlies the cuff and is bonded to it, such that on deflation the cuff may be collapsed upon it, thereby encouraging the cuff to pack flat.

33. A method of treating a patient using a device according to any one of claims 1 to 32.
